(19) United States
(12) Patent Application Publication
(10) Pub. No.: US 2026/0014000 A1
(43) Pub. Date: Jan. 15, 2026

(54) USE OF MOBILE COMPUTING DEVICE TO CHARGE IMPLANTABLE MEDICAL DEVICE

(71) Applicant: Boston Scientific Scimed, Inc., Maple Grove, MN (US)

(72) Inventors: Matthew Philip Dion, Saint Francis, MN (US); Keith R. Maile, New Brighton, MN (US); Joey Chen, Stevenson Ranch, CA (US); Brian Thomas Swanson, Roseville, MN (US)

(21) Appl. No.: 19/261,376

(22) Filed: Jul. 7, 2025

Related U.S. Application Data
(60) Provisional application No. 63/668,952, filed on Jul. 9, 2024.

(51) Int. Cl. A61F 2/48; A61F 2/26; H02J 7/00; H02J 50/10; H02J 50/90

(52) U.S. Cl. CPC A61F 2/482; A61F 2/26; H02J 7/00712; H02J 50/10; H02J 50/90; A61F 2250/0001

(57) ABSTRACT

A charging system for charging a battery of an implantable medical device, includes: a charger coil assembly having a transmission coil configured to generate an alternating magnetic field to inductively transfer electrical energy from the charger coil assembly to the battery of the implantable medical device; and a mobile computing device that is electrically coupled to the charger coil assembly and where the mobile computing device includes: an energy storage device, a memory configured for storing executable instructions, and a processor configured for executing the executable instructions to control the transfer of electrical energy from the energy storage device to the transmission coil for inductive transfer from the charger coil assembly to the battery of the implantable medical device.

### CROSS-REFERENCE TO RELATED APPLICATION

This application claims priority to U.S. Provisional Patent Application No. 63/668,952, filed on Jul. 9, 2024, entitled “USE OF MOBILE COMPUTING DEVICE TO CHARGE IMPLANTABLE MEDICAL DEVICE”, the disclosure of which is incorporated by reference herein in its entirety.

### TECHNICAL FIELD

This disclosure relates generally to bodily implants, and more specifically to the use of a mobile computing device to charge a bodily implant.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically controlled manifold providing for the transfer of fluid within the implantable fluid-operated device.

The use of the electronic control system may provide for more accurate actuation and control of the flow of fluid between components of the inflatable device, thus improving performance and efficacy of the device, as well as patient comfort and safety. The electronic control system may include one or more electronically-operated pumps and one or more valves to control the flow of fluid in the system, and the pumps and valves may be operated by way of piezoelectric elements associated with the pumps and valves. Electronically-operated pumps and valves require a source of power to operate, and therefore an electronically-operated implantable device can include a battery that stores power for use by the pumps and valves in the device. The battery can be a rechargeable battery that can be recharged by the transcutaneous transfer of power to the battery in the implantable device.

Charging devices used to recharge a battery in the implantable device can be complex, and therefore a need exists simplify the charging devices, reduce the number of components needed to operate the charging device, while providing a high level of functionality and usability for a user.

### SUMMARY

According to a general aspect, the techniques described herein relate to a charging system for charging a battery of an implantable medical device, where the charging system includes: a charger coil assembly having a transmission coil configured to generate an alternating magnetic field to inductively transfer electrical energy from the charger coil assembly to the battery of the implantable medical device; and a mobile computing device that is electrically coupled to the charger coil assembly and where the mobile computing device includes: an energy storage device, a memory configured for storing executable instructions, and a processor configured for executing the executable instructions to control the transfer of electrical energy from the energy storage device to the transmission coil for inductive transfer from the charger coil assembly to the battery of the implantable medical device.

Implementations can include one or more of the following features, alone or in any combination with each other.

In an example implementation, the mobile computing device is a mobile phone.

In an example implementation, the charging system further includes a cable that electrically couples the mobile computing device to the charger coil assembly.

In an example implementation, the mobile computing device includes a USB port and the charger coil assembly includes a USB port, and the cable includes a USB cable that electrically couples the USB port of the mobile computing device to the USB port of the charger coil assembly.

In an example implementation, the mobile computing device further includes voltage control circuitry configured for controlling a voltage on a power delivery line of the USB port of the mobile computing device to control a power that is transmitted to the implantable medical device by the charger coil assembly, where the mobile computing device further includes current control circuitry configured for controlling a current on a power delivery line of the USB port of the mobile computing device to control the power that is transmitted to the implantable medical device by the charger coil assembly.

In an example implementation, the executable instructions include app instructions for executing an app that controls a charging operation involving a transfer of energy from the energy storage device to the battery, and wherein execution of the app instructions by the processor causes processor to control the voltage control circuitry and the current control circuitry to maintain a predetermined amount of power transmitted from the mobile computing device to the implantable medical device through the charger coil assembly.

In an example implementation, execution of the app instructions by the processor further causes the processor to communicate commands over the USB port of the mobile computing device to a pulse width modulation controller of the charger coil assembly to control the amount of power transmitted from the mobile computing device to the implantable medical device through the charger coil assembly.

In an example implementation, the charger coil assembly includes a plurality of sensing coils configured to determine a proximity and alignment of a receiving coil of the implantable medical device relative to the transmission coil, and the processor is configured to control the voltage control circuitry and the current control circuitry to maintain the predetermined amount of power transmitted from the mobile computing device to the implantable medical device through the charger coil assembly based on the determined proximity and alignment.

In an example implementation, the mobile computing device further includes a display, and the executable instructions include app instructions for executing an app that controls a charging operation involving a transfer of energy from the energy storage device to the battery, and execution of the app instructions by the processor causes the mobile computing device to provide a graphical user interface on the display and the graphical user interface provides information to a user of the implantable medical device about at least one of: how much charge has been provided to the implantable medical device during the charging operation, an estimated end time of the charging operation, a current state of charge of the implantable medical device, or an amount of current or power being supplied to the implantable medical device at a particular time during the charging.

In an example implementation, the charger coil assembly does not include any optical user interface that provides information to a user of the implantable medical device about how much charge has been provided to the implantable medical device during the charging operation, an estimated end time of the charging operation, a current state of charge of the implantable medical device, or an amount of current or power being supplied to the implantable medical device at a particular time during the charging.

In some aspects, the techniques described herein relate to a method of charging a battery of an implantable medical device, the method including: electrically coupling a mobile computing device to a charger coil assembly that includes a transmission coil configured to generate an alternating magnetic field to inductively transfer electrical energy from the charger coil assembly to the battery of the implantable medical device, where the mobile computing device includes an energy storage device, a memory configured for storing executable instructions, and a processor configured for executing the executable instructions; and executing the stored executable instructions to control the transfer of electrical energy from the energy storage device to the transmission coil for inductive transfer from the charger coil assembly to the battery of the implantable medical device.

Implementations can include one or more of the following features, alone or in any combination with each other.

In an example implementation, the mobile computing device is a mobile phone.

In an example implementation, coupling the mobile computing device to the charger coil assembly includes connecting a cable between the mobile computing device and the charger coil assembly.

In an example implementation, the mobile computing device includes a USB port and the charger coil assembly includes a USB port, and the cable includes a USB cable that electrically couples the USB port of the mobile computing device to the USB port of the charger coil assembly.

In an example implementation, the method further includes controlling a voltage on a power delivery line of the USB port of the mobile computing device to control a power that is transmitted to the implantable medical device by the charger coil assembly; and controlling a current on a power delivery line of the USB port of the mobile computing device to control the power that is transmitted to the implantable medical device by the charger coil assembly.

In an example implementation, the method further includes: executing an app on the mobile computing device, where the app controls a charging operation involving a transfer of energy from the energy storage device to the battery; and controlling the transfer of electrical energy to maintain a predetermined amount of power transmitted from the mobile computing device to the implantable medical device through the charger coil assembly.

In an example implementation, the method further includes communicating commands over the USB port of the mobile computing device to a pulse width modulation controller of the charger coil assembly to control the amount of power transmitted from the mobile computing device to the implantable medical device through the charger coil assembly.

In an example implementation, the method further includes determining a proximity and alignment of a receiving coil of the implantable medical device relative to the transmission coil, based on signals from a plurality of sensing coils of the charger coil assembly, and maintaining the predetermined amount of power transmitted from the mobile computing device to the implantable medical device through the charger coil assembly based on the determined proximity and alignment.

In an example implementation, the mobile computing device further includes a display, and the method further includes: providing a graphical user interface on the display, wherein the graphical user interface provides information to a user of the implantable medical device about at least one of: how much charge has been provided to the implantable medical device during a charging operation, an estimated end time of the charging operation, a current state of charge of the implantable medical device, or an amount of current or power being supplied to the implantable medical device at a particular time during the charging.

In an example implementation, the charger coil assembly does not include any optical user interface that provides information to a user of the implantable medical device about how much charge has been provided to the implantable medical device during the charging operation, an estimated end time of the charging operation, a current state of charge of the implantable medical device, or an amount of current or power being supplied to the implantable medical device at a particular time during the charging.

### BRIEF DESCRIPTION OF THE DRAWINGS

FIG. 1 is a block diagram of an implantable fluid-operated inflatable device.

FIG. 2 illustrates a system including an example implantable fluid-operated inflatable device.

FIG. 3 is a schematic diagram of a fluidic architecture of an implantable fluid-operated inflatable device.

FIG. 4 is a perspective view of an example implantable medical device alongside a charging system.

FIG. 5 is a schematic block diagram of a charging system that is configured to recharge a rechargeable battery of an implantable medical device, wherein the charging system includes a charger coil assembly and a mobile computing device.

FIG. 6 is a flowchart of an example process for charging a battery of an implantable medical device.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants that include rechargeable batteries and to charging devices that are used to recharge the batteries of the bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure. Although the devices and techniques described herein are applicable to a variety to bodily implants that include rechargeable batteries, including, for example, electrical stimulation systems, pacemakers, cardiac defibrillators, artificial sphincters, cochlear implants, drug delivery systems, etc., the devices and techniques are described herein primarily in the context oof an implantable fluid-operated inflatable device.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically-operated.

For example, the pumps and/or valves of the fluid control system can be electronically-operated by the fluid control system to control the pressure of, and the flow of fluid in, parts of the fluid-operated inflatable device. An electronically-operated fluid control system, in accordance with implementations described herein, can include a plurality of electromechanical devices, such as, for example, piezoelectric devices that operate as pumps or as valves in the system. One or more controllers can control the electromechanical devices. An external charging system can provide energy to one or more rechargeable batteries in the implantable device. In some implementations, the external charging system can include a mobile computing device (e.g., a mobile phone), which supplies electrical power for recharging the battery(ies) of the implantable device, and a charger coil assembly connected to the mobile computing device that transmits the electrical power to the implanted implantable derive.

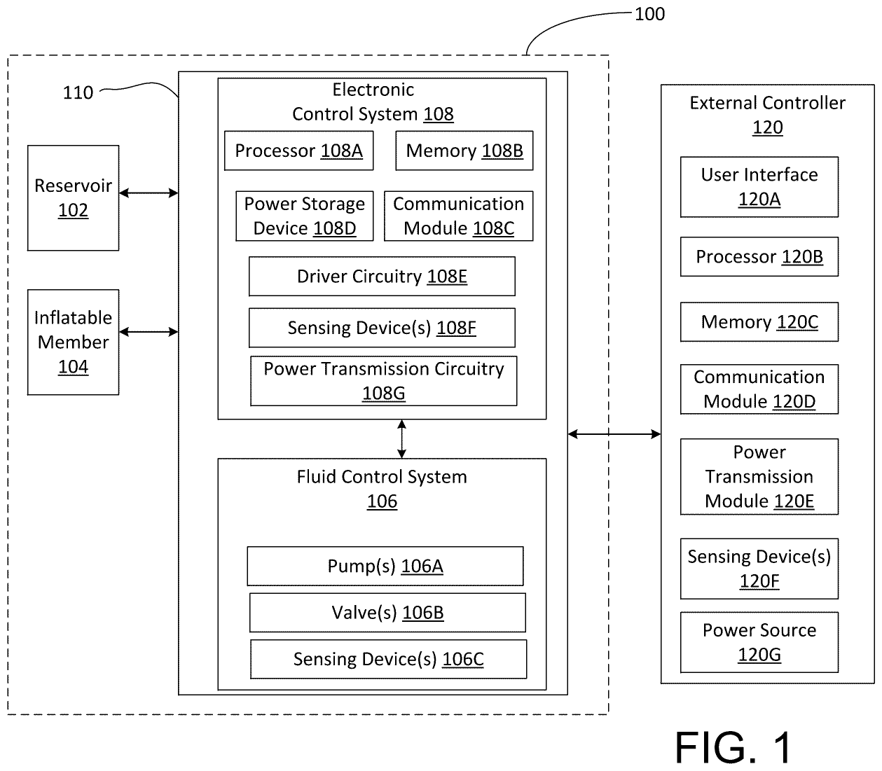

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104.

The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, an energy storage device 108D (e.g., a battery), electronic driver circuitry 108E, sensing devices 108F, such as, for example, voltage measurement circuitry, current measurement circuitry, an accelerometer, and other such components configured to provide for the monitoring, operation, and control of the implantable fluid-operated inflatable device 100.

The electronic control system can include energy transmission circuitry 108G configured for receiving power from an external controller 120, for example, though inductive coupling of electrical energy from the external controller to the electronic control system. In some examples, the communication module 108C of the electronic control system 108 may provide for communication with one or more external devices such as, for example, the external controller 120. Some of the components (for example, the processor 108A, the memory 108B, the communication module 108C, the energy storage device 108D, the electronic driver circuitry 108E, the sensing devices 108F, and the energy transmission circuitry 108G) of the inflatable device 100 can be positioned on one or more circuit boards or similar carriers within a sealed housing of an implantable device.

In some examples, the external controller 120 includes components such as, for example, a user interface 120A, a processor 120B, a memory 120C, a communication module 120D, an energy transmission module 120E, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface 120A, and to transmit the user inputs, for example, via the communication module 120D, to the electronic control system 108 for processing, operation, and control of the inflatable device 100. Similarly, the electronic control system 108 may, via the respective communication modules 108C, transmit operational information to the external controller 120. This may allow operational status of the inflatable device 100 to be provided, for example, through the user interface of the external controller 120, to the user, may allow diagnostics information to be provided to a physician, a technician, and the like.

In one implementation, an antenna included in the communication module 108C is capable of receiving signals (e.g., RF signals, such as Bluetooth® signals) from a communication module 120D of the external controller 120. Instructions stored in memory 120C can be executed by processor 120B to transmit signals over the communication module 120D to the implantable device.

Signals sent from the external controller 120 to the implantable device 100 via the communications modules 120D. 108C can be used to modify or otherwise direct the operation of the implantable device. For example, the signals may be used to modify the waveforms of electrical energy provided by the electronic control system 108 to the fluid control system 106, including, for example, modifying one or more of the waveform's frequency, amplitude, shape, and duration. The signals may also direct the implantable device 100 to cease operation, to start operation, to start charging the energy storage device 108D, or to stop charging the energy storage device 108D.

The communication module 108C of the implantable device 100 may include an antenna configured for transmitting signals back to the communication module 120D. For example, the implantable device 100 may transmit signals indicating whether the implantable device 100 is operating properly or not or indicating when the battery needs to be charged or the level of charge remaining in the battery.

In some examples, the energy transmission module 120E of the external controller 120 provides electrical power for charging of the components of the internal electronic control system 108. The electrical power may be provided from the energy source 120G of the external controller to the energy storage device 108D of the implantable device 100 through inductive coupling of an antenna that is part of the energy transmission module 120E of the external controller 120 to an antenna that is part of the energy transmission circuitry 108G. The antenna of the energy transmission module 120E of the external controller 120 can be positioned over the skin of the user in proximity with the antenna of the energy transmission circuitry 108G of the implantable device 100 to facilitate the transmission of energy from the external controller to the implantable device. Examples of such arrangements can be found in U.S. Pat. No. 6,895,280, which is incorporated herein by reference.

In some implementations the external controller 120 can include sensing devices 120F such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be implanted internally into the body of the patient. In some implementations, the electronic control system 108 and the fluid control system 106 are coupled in, or incorporated into, a housing 110. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

In some examples, electronic monitoring and control of the implantable fluid-operated inflatable device 100 may provide for improved patient control of the device, improved patient comfort, improved patient safety, and the like. In some examples, electronic monitoring and control of the implantable fluid-operated inflatable device 100 may afford the opportunity for tailoring of the operation of the inflatable device 100 by a physician without further surgical intervention. The fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

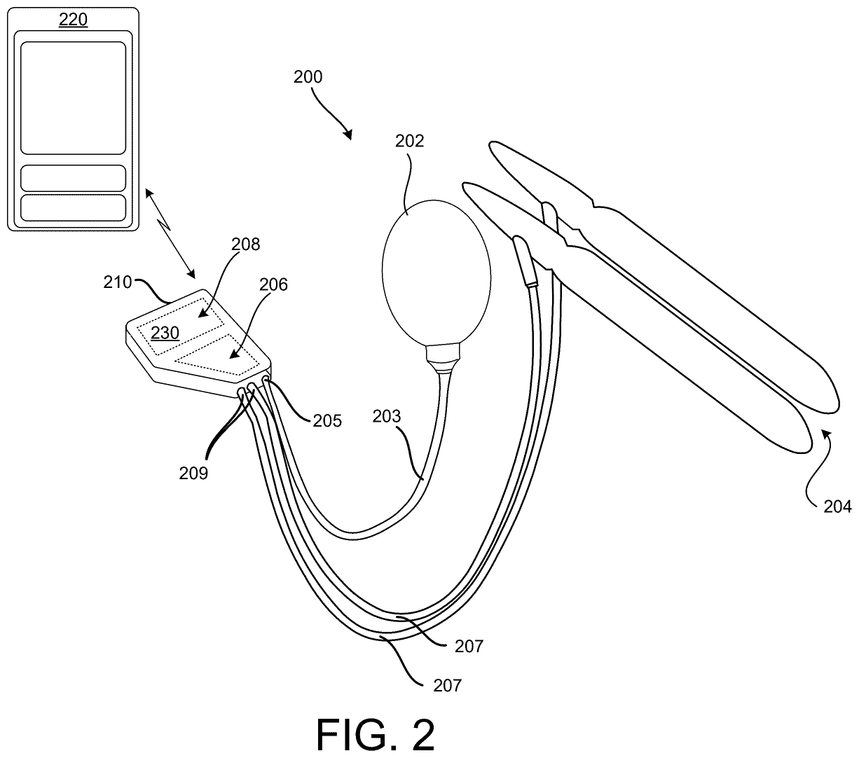

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid-operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, when the external controller 220 includes a mobile computing device (e.g., a mobile phone) an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by an energy transmission module of the external controller 220.

The principles to be described herein are applicable to the example implantable fluid-operated inflatable device, in the form of the example inflatable penile prostheses shown in FIG. 2, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump devices and one or more valve devices and one or more sensing devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

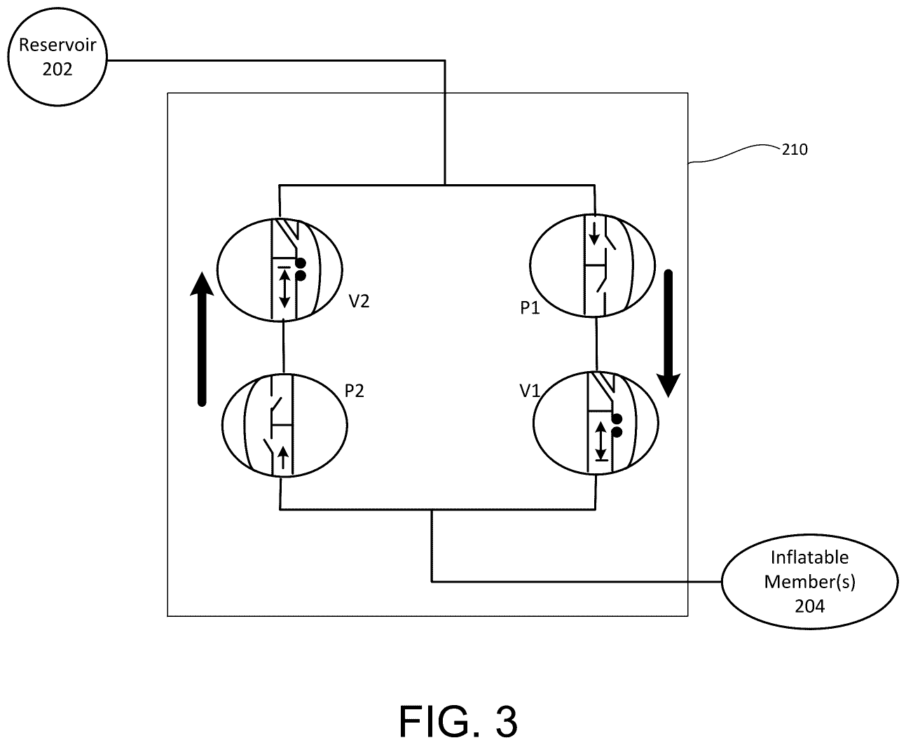

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

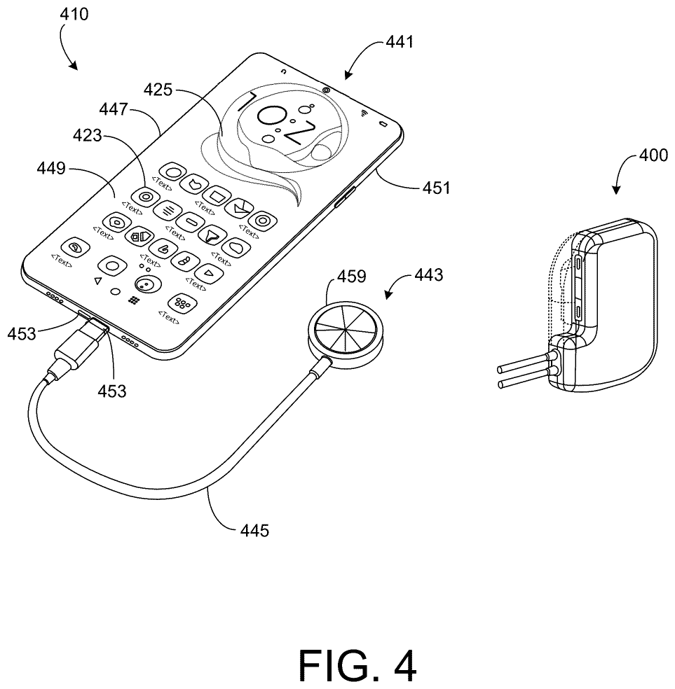

FIG. 4 is a perspective view of an example implantable medical device 400 alongside a charging system 410. The charging system 410 includes a mobile computing device (e.g., a mobile phone, a smart phone, a tablet, a laptop) 441 and a charger coil assembly (“coil assembly”) 443 that is electrically connected to the mobile computing device by a cord (e.g., by a USB cord) 445. The coil assembly 443 includes a coil disposed in a coil housing 459. The mobile computing device 441 includes an energy source 447 (e.g., one or more batteries) and an electronics subassembly 449 disposed in a housing 451. One or more electrical ports 453 (e.g., a USB port for receiving a USB cable for recharging the battery 447 of the mobile computing device and/or for providing energy from the battery 447 through the coil assembly 443 to the implantable device 400) may extend through the controller housing. As shown in FIG. 4, a USB cable 445 is connected to the mobile computing device 441 by way of a USB port 453.

In at least some embodiments, a charger cable 445 couples the coil assembly 443 to the mobile computing device 441. It may be advantageous to physically separate the coil assembly from other components of the charging system 410 (e.g., from the mobile computing device), as internal components of the coil assembly 443 may reach temperatures that are potentially dangerous for one or more components of the charger (e.g., the electronics subassembly, the optional power source, or other components) during operation of the charging system.

The mobile computing device 441 includes hardware (e.g., a one or more processors, drivers, memories, displays, speakers, etc.), firmware, and software (e.g., executable code) to enable the operation of one or more mobile device applications (“apps”) on the mobile computing device. A mobile device application can be downloaded from an online marketplace (e.g., Apple's App Store or Google's Play Store) for execution on a mobile device, such as, for example, a mobile phone. For example, an app can be launched by selecting an app icon 423 from a display screen 425 of the mobile computing device 441. At least one app running on the mobile computing device 441 can be configured to control the charging of an energy storage device of the implantable medical device 400 with energy stored in an energy source 447 of the mobile computing device. The at least one app can include a user interface through which a user (e.g., a patient, a clinician, etc.) interacts with the app to control the charging and the operation of the implantable device. The user interface can include one or more controls, such as a START/STOP charging control. The user interface may further include one or more indicators, such as an alignment indicator, a power-level indicator, and a charging status indicator, etc. In at least some embodiments, the one or more indicators can include at least one visual indicator, suitable for being seen by a patient during operation of the charger (e.g., during a charging session). In at least some embodiments, the one or more indicators include at least one aural indicator, such as one or more speakers configured to produce one or more audible signals suitable for being heard by a patient during operation of the charger.

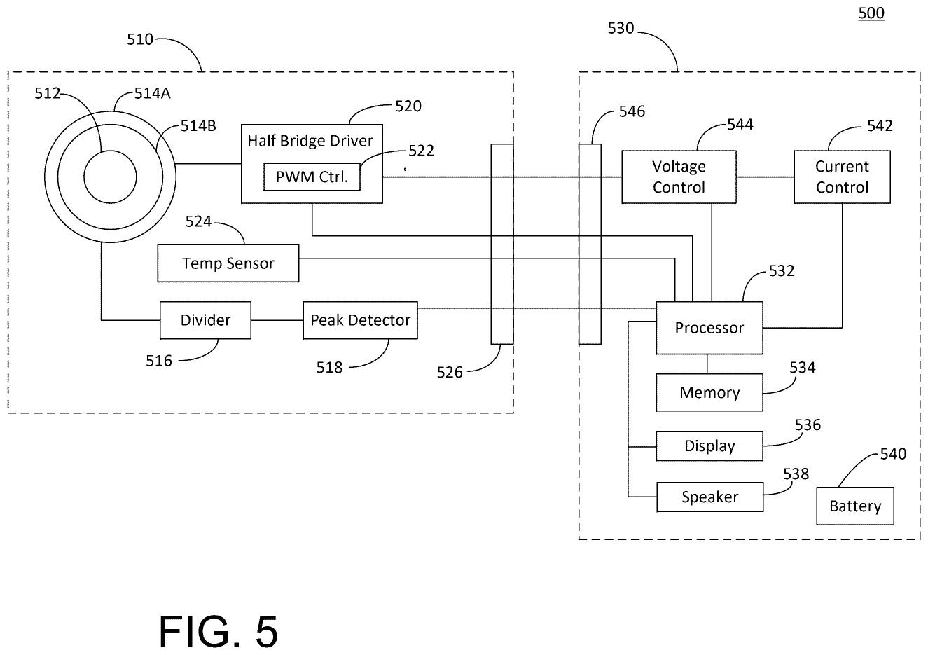

FIG. 5 is a schematic block diagram of a charging system 500 that is configured to recharge a rechargeable battery of an implantable medical device. The charging system 500 includes a dedicated charger coil assembly 510 and a mobile computing device (e.g., a smart phone, a tablet, a laptop) 530. The mobile computing device 530 is configured to control the charger coil assembly 510, so that the charger coil assembly 510 can transfer energy from the mobile computing device to the implantable medical device through an inductive charging process. The mobile computing device 530 and the charger coil assembly 510 can be coupled to each other through standard hardware and software protocols. For example, the mobile computing device 530 and the charger coil assembly 510 each can include a USB port 546, 526, respectively, and a USB-compliant cable can be plugged into the respective USB ports 546, 526 to couple the two devices. When coupled to each other, for example, over a USB compliant cable, the mobile computing device 530 and the charger coil assembly 510 can exchange communication signals and power through the coupling.

The charger coil assembly 510 produces an alternating current in a transmission coil 512, and the alternating current generates a varying magnetic field, which, in turn, creates an alternating current in a receiving coil of the implantable medical device when the transmission coil 512 is proximate to the receiving coil. Within the implantable medical device, the alternating current can be used to recharge a battery of the implantable medical device. To determine the relative proximity and alignment of the transmission coil 512 and the receiving coil of the implantable medical device, sensing coils 514A, 514B (which, in some implementations, can be concentric with each other and/or with the transmission coil 512) each can detect a signal from the receiving coil, and after dividing down the detected signals in a divider 516, the signals can be compared by a detector circuitry 518. Based on the amplitudes of the detected signals and their relative values, the proximity of the transmission coil 512 to the receiving coil in the alignment of the transmission coil 512 with the receiving coil can be determined.

In some implementations, the charger coil assembly 510 includes a temperature sensor 524 that generates a signal based on the temperature of the sensor in the assembly. The signal can be communicated to a processor 532 of the mobile computing device and can be used to control a process of charging an implantable medical device with power provided through the charger coil assembly 510. For example, if the temperature sensor 524 indicates a temperature that exceeds a threshold amount, the processor 532 may take action to halt the charging process until the temperature registered by the temperature sensor falls below the threshold temperature.

Energy to drive the alternating current in the transmission coil 512 can be provided from an energy source (e.g., a battery) 540 in the mobile computing device to a driver circuit 520 in the charger coil assembly 510. In some implementations, the driver circuit 520 includes a half-bridge circuit that generates the alternating current in the transmission coil 512. A pulse width modulation circuit 522 within the driver 520 can be used to control the power supplied to the transmission coil, for a given voltage and current that are supplied by the mobile computing device 530 to the charger coil assembly 510.

The mobile computing device 530 includes a processor 532 configured for processing and executing instructions, for example, instructions stored in a memory 534. In some implementations, the memory 534 can include instructions for executing a mobile device application (e.g., an “app”) that is designed to control the re-charging of a battery of an implantable medical device with energy supplied from a battery 540 in the mobile computing device and transmitted to the implantable medical device by the charger coil assembly 510.

The mobile device application can be executed by the processor 532, and the execution of the app can cause the mobile computing device 530 to generate a graphical user interface on a display 536. The graphical user interface can provide information to a user of the implantable medical device about a charging operation of the implantable medical device, such as, for example, how much charge has been provided to the implantable medical device during a charging operation, an estimated end time of the charging operation, a current state of charge of the implantable medical device, an amount of current or power being supplied to the implantable medical device at a particular time during the charging operation, etc. With this information being provided to the user through the graphical user interface of the mobile computing device 530, the charger coil assembly need not include any optical user interface to provide such information to the user, and, in some implementations, the charger coil assembly does not include any optical user interface for providing information to the user.

The mobile computing device 530 includes one or more audio speakers 538 that can provide audible information to a user of the implantable medical device about the charging operation. For example, execution of the app can cause the speaker 538 of the mobile computing device 530 to generate audible sounds that can indicate different aspects of the charging process, such as, for example, when the charging operation is complete, when an amount of charge in the implantable medical device exceeds a threshold amount, etc.

Various combinations of software, firmware, and hardware in the mobile computing device 530 can be used to control the charging operation of a battery of the implantable medical device. For example, the USB standard includes a USB Power Delivery (USB PD) specification that standardizes the protocols for providing power over a USB connection between devices. The USB PD Revision 3.1 specification announced in 2021 specifies that the voltage of a charging signal provided on a USB line can be selected to be one of a number of different fixed voltages, up to 48 volts, and that the current supplied on the line can range from zero to 5 Amps. Execution of the app can cause the processor 532 to exert control over the voltage control circuit 544 that controls the voltage of the USB charging signal and to exert control over the current control circuit 542 that controls the current of the charging signal. Control over the voltage control circuit and control over the current control circuit can be used to maintain a predetermined amount of power transmitted from the mobile computing device to the implantable medical device through the charger coil assembly.

Control over the power provided to the implantable medical device from the transmission coil 512 of the charger coil assembly 510 can be achieved through the loop shown in FIG. 5. For example, execution of the app running on the mobile computing device 530 can specify an amount of power to transmit to the battery of the implantable medical device. The processor 532 can control the voltage and current of the charging signal through the voltage control circuit 544 and the current control circuit 542, respectively. In addition, the processor 532 can communicate commands over the USB interface to the pulse width modulation controller 522 to control the power provided to the implantable medical device through the transmission coil 512. Signals generated by the sense coils 514A, 514B can be used to determine a proximity and alignment of the receiving coil of the implantable medical device relative to the transmission coil 512, and these signals can be communicated over the USB interface to the processor 532 which can use the signals to determine what percentage of power provided from the mobile computing device 530 is received by the implantable medical device. Based on this determination, the processor can adjust the power that is provided to the transmission coil 512 through its control of the current control circuit 542, the voltage control circuit 544, and the pulse width modulation control 522.

Thus, the bulk of the processing and control of the charging signal can be offloaded from the charger coil assembly 510 onto the existing resources of the mobile computing device 530. In addition, the resources of the mobile computing device 530 can be leveraged to provide a relatively sophisticated user interface into the charging operation, for example, through an app running on the mobile computing device, where the user interfaces rendered to a user through the display 536 and/or audio speakers 538 of the mobile computing device 530. Because of this, the charge coil assembly 510 can be relatively unsophisticated, in that certain circuits, such as, the voltage control circuit 544 and the current control circuit 542 need not be included in the charge coil assembly, and the charge coil assembly need not include a sophisticated processor, or a display or speakers to render a user interface.

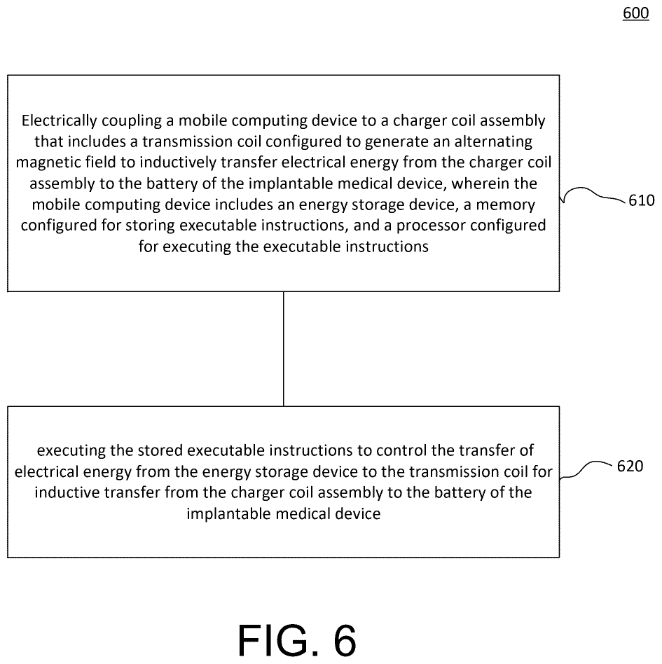

FIG. 6 is a flowchart of an example process 600 for charging a battery of an implantable medical device. In the process 600, at step 610, a mobile computing device is electrically coupled to a charger coil assembly that includes a transmission coil configured to generate an alternating magnetic field to inductively transfer electrical energy from the charger coil assembly to the battery of the implantable medical device, where the mobile computing device includes an energy storage device, a memory configured for storing executable instructions, and a processor configured for executing the executable instructions. At step 620, the stored executable instructions are executed to control the transfer of electrical energy from the energy storage device to the transmission coil for inductive transfer from the charger coil assembly to the battery of the implantable medical device.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A charging system for charging a battery of an implantable medical device, the charging system comprising:
a charger coil assembly including a transmission coil configured to generate an alternating magnetic field to inductively transfer electrical energy from the charger coil assembly to the battery of the implantable medical device; and
a mobile computing device that is electrically coupled to the charger coil assembly and that includes:
an energy storage device,
a memory configured for storing executable instructions; and
a processor configured for executing the executable instructions to control the transfer of electrical energy from the energy storage device to the transmission coil for inductive transfer from the charger coil assembly to the battery of the implantable medical device.

2. The charging system of claim 1, wherein mobile computing device is a mobile phone.

3. The charging system of claim 1, further comprising a cable that electrically couples the mobile computing device to the charger coil assembly.

4. The charging system of claim 3, wherein the mobile computing device includes a USB port and the charger coil assembly includes a USB port, and wherein the cable includes a USB cable that electrically couples the USB port of the mobile computing device to the USB port of the charger coil assembly.

5. The charging system of claim 4,
wherein the mobile computing device further includes voltage control circuitry configured for controlling a voltage on a power delivery line of the USB port of the mobile computing device to control a power that is transmitted to the implantable medical device by the charger coil assembly,
wherein the mobile computing device further includes current control circuitry configured for controlling a current on a power delivery line of the USB port of the mobile computing device to control the power that is transmitted to the implantable medical device by the charger coil assembly.

6. The charging system of claim 5,
wherein the executable instructions include app instructions for executing an app that controls a charging operation involving a transfer of energy from the energy storage device to the battery, and wherein execution of the app instructions by the processor causes processor to control the voltage control circuitry and the current control circuitry to maintain a predetermined amount of power transmitted from the mobile computing device to the implantable medical device through the charger coil assembly.

7. The charging system of claim 6, wherein execution of the app instructions by the processor further causes the processor to communicate commands over the USB port of the mobile computing device to a pulse width modulation controller of the charger coil assembly to control the amount of power transmitted from the mobile computing device to the implantable medical device through the charger coil assembly.

8. The charging system of claim 6,
wherein the charger coil assembly includes a plurality of sensing coils configured to determine a proximity and alignment of a receiving coil of the implantable medical device relative to the transmission coil, and
wherein the processor is configured to control the voltage control circuitry and the current control circuitry to maintain the predetermined amount of power transmitted from the mobile computing device to the implantable medical device through the charger coil assembly based on the determined proximity and alignment.

9. The charging system of claim 1,
wherein the mobile computing device further includes a display, and
wherein the executable instructions include app instructions for executing an app that controls a charging operation involving a transfer of energy from the energy storage device to the battery, and wherein execution of the app instructions by the processor causes the mobile computing device to provide a graphical user interface on the display and the graphical user interface provides information to a user of the implantable medical device about at least one of: how much charge has been provided to the implantable medical device during the charging operation, an estimated end time of the charging operation, a current state of charge of the implantable medical device, or an amount of current or power being supplied to the implantable medical device at a particular time during the charging.

10. The charging system of claim 9,
wherein the charger coil assembly does not include any optical user interface that provides information to a user of the implantable medical device about how much charge has been provided to the implantable medical device during the charging operation, an estimated end time of the charging operation, a current state of charge of the implantable medical device, or an amount of current or power being supplied to the implantable medical device at a particular time during the charging.

11. A method of charging a battery of an implantable medical device, the method comprising:
electrically coupling a mobile computing device to a charger coil assembly that includes a transmission coil configured to generate an alternating magnetic field to inductively transfer electrical energy from the charger coil assembly to the battery of the implantable medical device, wherein the mobile computing device includes an energy storage device, a memory configured for storing executable instructions, and a processor configured for executing the executable instructions;
executing the stored executable instructions to control the transfer of electrical energy from the energy storage device to the transmission coil for inductive transfer from the charger coil assembly to the battery of the implantable medical device.

12. The method of claim 11, wherein the mobile computing device is a mobile phone.

13. The method of claim 11, wherein coupling the mobile computing device to the charger coil assembly includes connecting a cable between the mobile computing device and the charger coil assembly.

14. The method of claim 13, wherein the mobile computing device includes a USB port and the charger coil assembly includes a USB port, and wherein the cable includes a USB cable that electrically couples the USB port of the mobile computing device to the USB port of the charger coil assembly.

15. The method of claim 14, further comprising:
controlling a voltage on a power delivery line of the USB port of the mobile computing device to control a power that is transmitted to the implantable medical device by the charger coil assembly; and
controlling a current on a power delivery line of the USB port of the mobile computing device to control the power that is transmitted to the implantable medical device by the charger coil assembly.

16. The method of claim 15, further comprising:
executing an app on the mobile computing device, wherein the app controls a charging operation involving a transfer of energy from the energy storage device to the battery; and
controlling the transfer of electrical energy to maintain a predetermined amount of power transmitted from the mobile computing device to the implantable medical device through the charger coil assembly.

17. The method of claim 16, further comprising communicating commands over the USB port of the mobile computing device to a pulse width modulation controller of the charger coil assembly to control the amount of power transmitted from the mobile computing device to the implantable medical device through the charger coil assembly.

18. The method of claim 16,
determining a proximity and alignment of a receiving coil of the implantable medical device relative to the transmission coil, based on signals from a plurality of sensing coils of the charger coil assembly, and
maintaining the predetermined amount of power transmitted from the mobile computing device to the implantable medical device through the charger coil assembly based on the determined proximity and alignment.

19. The method of claim 11, wherein the mobile computing device further includes a display, and the method further comprises:
providing a graphical user interface on the display, wherein the graphical user interface provides information to a user of the implantable medical device about at least one of: how much charge has been provided to the implantable medical device during a charging operation, an estimated end time of the charging operation, a current state of charge of the implantable medical device, or an amount of current or power being supplied to the implantable medical device at a particular time during the charging.

20. The method of claim 19,
wherein the charger coil assembly does not include any optical user interface that provides information to a user of the implantable medical device about how much charge has been provided to the implantable medical device during the charging operation, an estimated end time of the charging operation, a current state of charge of the implantable medical device, or an amount of current or power being supplied to the implantable medical device at a particular time during the charging.
